(19) United States
(12) Patent Application Publication
(10) Pub. No.: US 2016/0130543 A1
(43) Pub. Date: May 12, 2016

(54) Modular Microtube Network for Vascularized Organ-On-A-Chip Models

(71) Applicant: The Government of the United States of America, as represented by the Secretary of the Navy, Washington, DC (US)

(72) Inventors: Michael A. Daniele, Raleigh, NC (US); Steven A. Roberts, Fairfax, VA (US); Frances S. Ligler, Fuquay Varina, NC (US); André A. Adams, Burke, VA (US)

(21) Appl. No.: 14/936,860

(22) Filed: Nov. 10, 2015

Related U.S. Application Data
(60) Provisional application No. 62/077,375, filed on Nov. 10, 2014.

(51) Int. Cl. C12M 3/00; C12M 1/00; C12M 1/04; C12M 3/06

(52) U.S. Cl. CPC C12M 21/08; C12M 23/16; C12M 29/04; C12M 29/10; C12M 23/24

(57) ABSTRACT

A perfusable, microtube-based flow system for microdevice models of human tissues and organs includes a microfluidic chip with a network of microtubes passing through at least one common chamber space suitable to hold cells of an appropriate organ and/or a biocompatible support matrix. The microtube model network can be used to create model tissues and organs for study and research.

### CROSS-REFERENCE TO RELATED APPLICATIONS

This Application claims the benefit of U.S. Provisional Application No. 62/077,375 filed on Nov. 10, 2014, which is incorporated herein by reference in its entirety.

This Application is related to U.S. Pat. No. 9,157,060.

### BACKGROUND

A need exists for systems to perfuse microdevice models of tissues and organs. Such articles are often termed organ-on-a-chip systems.

### BRIEF SUMMARY

In a first embodiment, a model microtube network includes at least one microtube comprising a permeable wall surrounding a central hollow lumen open at opposing ends of the microtube, wherein the microtube has an outer diameter of between 5 and 8000 microns and the permeable wall has a thickness of between 0.1 and 250 microns with a pore size of from 0.1 microns to 500 microns; and a microfluidic chip having a chamber space defined by chamber walls, wherein the at least one microtube passes through the chamber space and through the chamber walls with the lumen open at each end outside the chamber space, such that a seal exists between the least one microtube and the chamber walls.

In another embodiment, a model microtube network includes at least one microtube comprising a permeable wall with living animal cells disposed within, the permeable wall surrounding a central hollow lumen open at opposing ends of the microtube, wherein the microtube has an outer diameter of between 5 and 8000 microns and the permeable wall has a thickness of between 0.1 and 250 microns with a pore size of from 0.1 microns to 500 microns; and a microfluidic chip having an input reservoir, an output reservoir, and a chamber space therebetween which holds extracellular matrix (ECM) material, the chamber space being defined by chamber walls separating the chamber space from the reservoirs, wherein the at least one microtube passes through the chamber space and through the chamber walls such that a seal exists between the least one microtube and the chamber walls, with the lumen open at each end to the input reservoir and output reservoir, respectively.

A further embodiment is a method of making a model microtube network, the method including providing at least one microtube comprising a permeable wall surrounding a central hollow lumen open at opposing ends of the microtube, wherein the microtube has an outer diameter of between 5 and 8000 microns and the permeable wall has a thickness of between 0.1 and 250 microns with a pore size of from 0.1 microns to 500 microns; and securing the at least one microtube in microfluidic chip having a chamber space defined by chamber walls, such that the at least one microtube passes through the chamber space and through the chamber walls with the lumen open at each end outside the chamber space.

Yet another embodiment is a system comprising the model microtube network of the first embodiment and and a pump operably connected to cause a fluid flow through the at least one microtube.

### BRIEF DESCRIPTION OF THE DRAWINGS

FIG. 1 is a conceptual schematic of exemplary systems in accordance with the present invention.

FIG. 2 is the simplified perspective view of one embodiment of the system of the present invention.

FIG. 3 is a photograph of the aligned microtubes and a photograph of a modular microtube manifold with impermeable barriers.

FIG. 4A is a schematic of one embodiment of the vascularized organ-on-a-chip microdevice containing the microtube manifold and a photograph of the manifold under operation. FIG. 4B is a photograph of a device showing passage of a marker fluid through the microtube manifold (contains 2 microtubes) across the cell culture chamber. The fluid inlet is connected via a thin channel to a larger deep well containing microtubules encapsulated in photopolymerized, impermeable rubber seals. Microtubule holders in the center of the device allow for alignment of microtubes, which traverses a middle well containing cells and appropriate support material.

FIG. 5A is a photograph of one embodiment of the vascularized organ-on-a-chip microdevice illustrating trypan blue fluid flow through the microdevice and showing the separation of the inlet, outlet, and tissue compartments. FIG. 5B shows a series of time lapse photographs in which the diffusion of trypan blue is illustrated as it is delivered across the microtube wall and into the cell culture chamber.

FIG. 6 is the perspective view of another embodiment of the system with multiple inlet and outlet distribution chambers.

### DETAILED DESCRIPTION

Definitions

Before describing the present invention in detail, it is to be understood that the terminology used in the specification is for the purpose of describing particular embodiments and is not necessarily intended to be limiting. Although many methods, structures and materials similar, modified, or equivalent to those described herein can be used in the practice of the present invention without undue experimentation, examples of preferred methods, structures and materials are described herein. In describing and claiming the present invention, the following terminology will be used in accordance with the definitions set out below.

As used in this specification and the appended claims, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

As used herein, the term “microtube” refers to a hollow tube or fiber with an outer diameter of between about 5 and 8000 microns.

As used herein, the term “manifold” refers to a microfluidic chip having a chamber space defined by walls and at least one microtube passing through the chamber space and through the walls thereof.

As used herein, the term “extracellular matrix” or “ECM” refers to biologically-derived and/or synthetic ECM material.

Overview

A goal of this invention is to provide a perfusable, microtube-based flow system for microdevice models of human tissues and organs. Such model tissues and organs can be perfused by tubes or fibers (termed microtubes) than can mimic blood vessels, glands, ducts, and other hollow structures that might be found in an organism. In a particular embodiment, a vascularized organ-on-a-chip system includes a microfluidic chip and a polymer microtube manifold. Techniques described herein are expected to increase the viability of thick tissue cultures, which otherwise suffer from necrosis due to the inability of nutrients diffuse efficiently through multiple layers of cells. The invention also expected to facilitate building and studying vascularized systems, which is a major shortcoming of most tissue-on-chip systems currently reported. By providing a three-dimensional (3D) culture system with embedded vasculature through which solutions can pass continually, the predictive value and physiological relevance of 3D organ models should be enhanced.

Suitable techniques relating to forming microfibers and tubes to mimic microvasculature and ducts are described in, e.g., U.S. Patent Application Publication 2014/0087466.

A significant challenge in generating viable 3D tissue and organ models is the recapitulation of vascular networks. Microfluidic perfusion systems have provided some improvement in mimicking native cellular environments, relative to conventional 3D culture methods. Typically, these systems rely on hydrostatic pressure to force fluid through a 3D matrix or cell culture chamber (Moraes C, et al., Annals of Biomedical Engineering. 2012;40:1211-27), or they incorporate channels to pass fluid through (Miller J S et al., Nat Mater. 2012; 11:768-74). Although some perfusion can be achieved by pressure-driven diffusion through a 3D matrix, the matrix must be kept quite thin, for example below 250 μm, to avoid necrosis from hypoxia. Moreover, the forces exerted by circulatory flow are not present in these systems, they have non-physiologically high liquid-to-cell ratios, and the cell types in complex co-culture systems are present in ratios that are not representative of normal physiology. Therefore, assay data derived using these models lack the patterns of biological interactions that would be found in native environments and are therefore fundamentally flawed.

Cell culture devices that better mimic in vivo vascular conditions are desirable. U.S. Pat. No. 5,612,188 issued to Shuler et al. describes multi-compartmental cell culture systems that create perfusion networks on a microdevice. The Shuler system creates a vasculature counterpart by connection of discrete chambers possibly containing vascular cell types, but the culture chambers rely on 2D models of tissue. The 2D model of vasculature lacks directionality, lumen formation, and physiologically representative mechanical stresses and cell-to-fluid ratios. A more relevant concept to generate a perfusable 3D culture utilized flow around a solid mandrel to provide fluid within a 3D cell culture to stimulate vascular microvessel formation (Neumann et al., U.S. Pat. No. 8,445,280B2). The Neumann method provides a route to forming endothelial tubes; however, this system is completely defined by the starting microdevice to generate the endothelial tubes and, it does not appear possible to be ported as a viable vascular system into an organ model.

The development of organized microtubes joined to the fluid reservoirs at each end as a manifold with multiple microtubes is expected to provide better, faster and more efficient perfusion for nutrient supply and waste removal to simulate microvasculature 3D organ models. Using microtubes that are biocompatible and can include endothelium and other cells characteristic of blood vessels increases the physiological relevance of the 3D organ models.

Description and Operation

Devices and methods are provided for an organ-on-a-chip device that permits cells to be maintained in vitro under conditions with perfusion parameters similar to those found in vascularized tissue. The specific microdevice geometry and components are designed to provide cellular interactions, liquid flow, and liquid residence parameters that correlate with those found for tissues or organs in vivo. The microtubes are designed to represent primary elements of the circulatory or lymphatic systems.

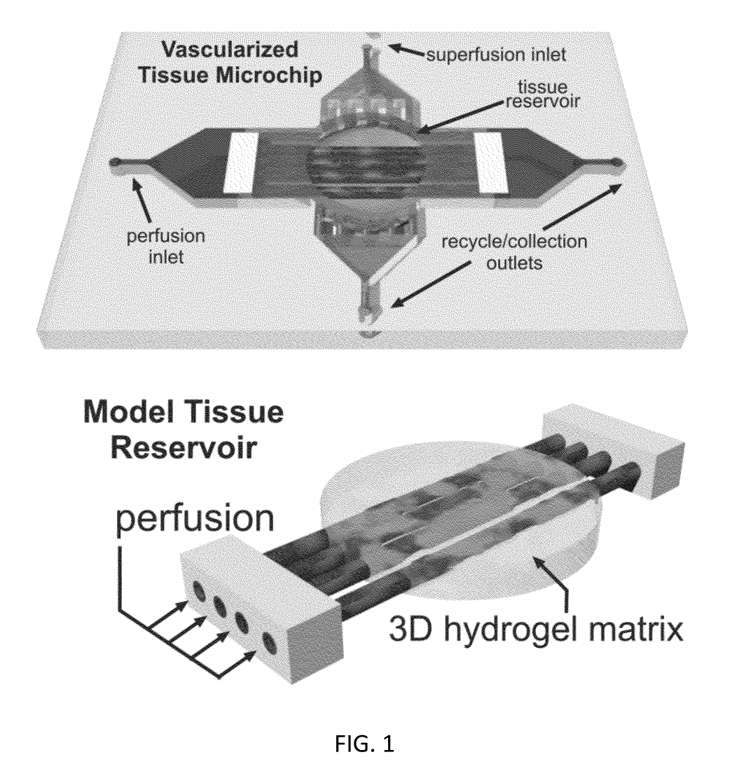

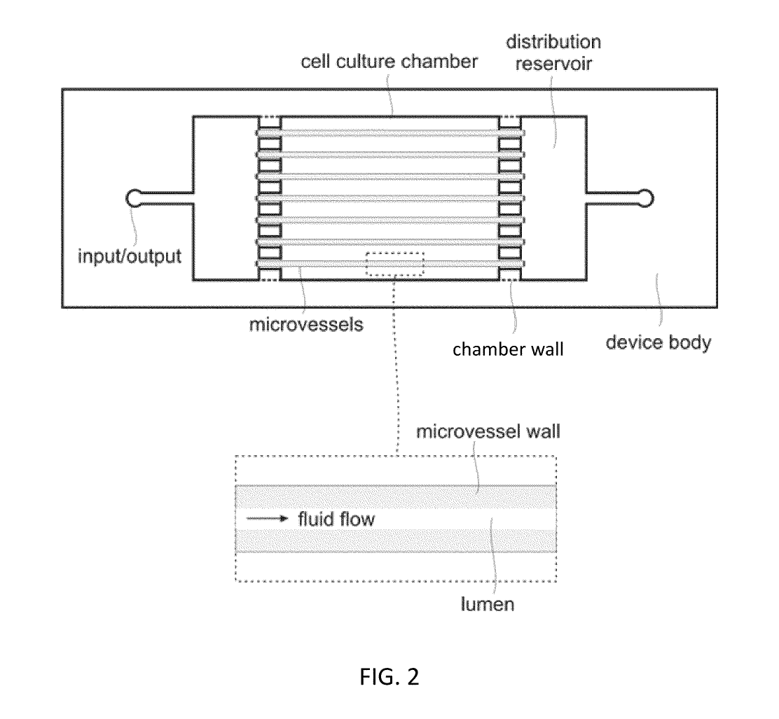

The organ-on-a-chip device comprises a microfluidic chip with a network of microtubes (sometimes called microvessels) passing through at least one common chamber space suitable to hold cells of an appropriate organ and/or a biocompatible support matrix. The central lumen passages in the microtubes can be operably connected to reservoirs operable to introduce and remove fluid from the microtubes. The microfluidic chip can be made of a substrate defining the chamber space and can optionally include at least two reservoirs separate from one another and from the cell culture chamber, or in the alternative the reservoirs can be provided separately from the microfluidic chip. In embodiments where the reservoirs and the chamber are on the same microfluidic chip, there is at least one microtube connecting at least two distribution reservoirs across the cell culture chamber (see exemplary FIGS. 1 and 2).

The fluid reservoirs have at least one input channel and at least one output channel and can be simple (for example, a dump tube) or more complex with internal structure to guide fluid flow to and from the microtubes. The chamber space can optionally have inlets and outlets suitable to perfuse the chamber.

Many structures for the device, including channels, reservoirs, chambers, walls, substrates, and assemblies thereof, may be cast from a mold or prepared by other suitable techniques including milling, etching, additive manufacturing, and combinations of these.

The microtubes can be prepared separately from the rest of the device. Examples of appropriate techniques for preparing appropriate microtubes via sheath flow techniques can be found in U.S. Patent Application Publication 2014/0087466, now U.S. Pat. No. 9,157,060. Embodiments of microtubes can have a wall consisting of a single polymer layer around a hollow lumen, or a wall of at least two concentric layers of polymer surrounding a central hollow lumen, wherein the fiber has an outer diameter of between 5 and 8000 microns and wherein each individual layer of polymer has a thickness of between 0.1 and 250 microns, wherein the wall is permeable with a pore size of from 0.1 microns to 500 microns

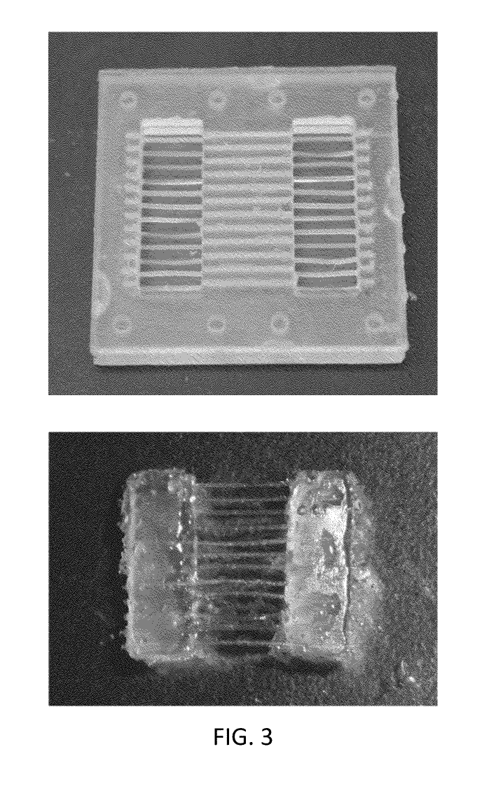

A microtube network crossing the cell culture chamber may be prepared by a process including the steps of: fabrication of the microtubes, aligning the microtubes in a microfluidic chip (see FIG. 3), and optionally encapsulating the end regions of the microtubes in an impermeable sealant where they pass through the chamber wall. The microtube openings connected are to a reservoir space, thus creating a manifold. In embodiments, the microtubes can be formed into the chamber wall without the use of a separate sealant. If so, preferably the microtubes are not appreciably crushed or collapsed during such process and the resistance through the microtube for fluid flow is lower than flow through any gap in the chamber wall into the cell culture chamber. In certain embodiments, either with or without the use of a separate sealant, the chamber walls are impermeable and without gaps other than the sealed passage of the microtubes, so that fluid from the input distribution reservoir is only delivered via the microtubes rather than by leakage into the chamber.

Suitable impermeable sealants include rubber and rubber-like polymers as well as other sealants known in the art. The microtubes move fluid from the distribution reservoirs across the culture chamber. The microfluidic chip can be cast from poly(dimethyl siloxane) or similar materials. The microtube walls are composed of a preferably semi-permeable material. The pore size of the microtube wall materials is tunable to provide passage of low and high molecular weight nutrients and may even be modified to accommodate possible movement of cells from the culture chamber into or out of the microtube. Pore size may be from 0.1 microns to 500 microns. The microdevice may be milled or molded from poly(methyl methacrylate) (PMMA), polystyrene, polycarbonate, polydimethylsiloxane (PDMS), polytetrafluoroethylene (PTFE), cyclic olefin copolymer, or another suitable material such as aluminum, silicon, or glass, and combinations of these. The performance of the microtube manifold and assessment of its functionality is based on desired parameters including input pressure, output pressure, overall flow rate, diffusion across the microtube wall, and intrinsic properties of the fluid.

The microtube and culture chamber can be seeded with almost any cell type. A description of cell-laden microtubes was disclosed in U.S. Patent Application Publication 2014/0087466, now U.S. Pat. No. 9,157,060. The culture chamber can be filled with a 3D scaffold material to simulate an extracellular matrix (ECM) such as MatriGel, collagen, gelatin, or the like. Cells that are characteristic of a particular tissue type or differentiate into tissues of interest can be introduced into the chamber with the scaffold material or afterward. For example, a device designed to provide a lung model may contain microtubes seeded with endothelial cells and a culture chamber seeded with a co-culture of alveolar and smooth-muscle cells, and may be fitted with multiple inlet and outlet distribution reservoirs and devices for gas exchange Using this system, many variables, including but not limited to, cell differentiation, cell-cell communication, cell and molecular transport, tissue size, tissue-to-fluid volume ratio, fluid residence time and hydrodynamic forces can be simulated and assessed.

Embodiments can have living animal cells disposed within the microtubes, within the chamber, and/or within the reservoir.

An example follows:

1. Microtubes comparable to mammalian capillaries are prepared by hydrodynamically focusing gelatin methacrylamide and polyethylene oxide polymers including human umbilical vein endothelial cells (HUVECs), mesenchymal stem cells and fibroblasts.

2. The microtubes are placed in a mold and poly(dimethylsiloxane) (PDMS) is polymerized around the ends to stabilize the microtubes in a manifold.

3. The ends of manifolds are cut to reveal the open ends of the microtubes.

4. The manifolds are inserted into reservoirs of culture media. The reservoirs are shaped to prevent leaking around the manifolds and extra PDMS added as a sealant if necessary.

5. Flow is established through the microtubes with a culture fluid containing endothelial growth factors. Pulsatile flow could be used to mimic vascular pumping.

6. The microtubes and manifolds are incorporated into the tissue culture chip. The central cavity is filled with a collagen or alginate hydrogel containing mesenchymal stem cells and osteoblast differentiation factors.

7. The cultures are maintained for several weeks. The endothelial differentiation factors are eliminated as soon as the endothelium covers the inner walls of the microtubes.

8. At one or more suitable time periods, e.g. from 3 days to 3 months, cultures may be fixed, stained and examined for differentiation, remodeling of the extracellular matrix, and establishment of higher order 3D structures within the tissue on chip. Drugs could be added and the effluent monitored to test for transport into the tissue. Fluid in the tissue could be monitored for processing of the drug and cells monitored for drug uptake and efficacy.

9. Tumor cells could be included in the vessels or tissue to monitor metastasis—or in the tissue to look at targeting of drugs introduced intravenously.

The process steps can be varied as understood by one of skill in the art. For example, the chamber can be filled with extracellular matrix material and/or cells at various times.

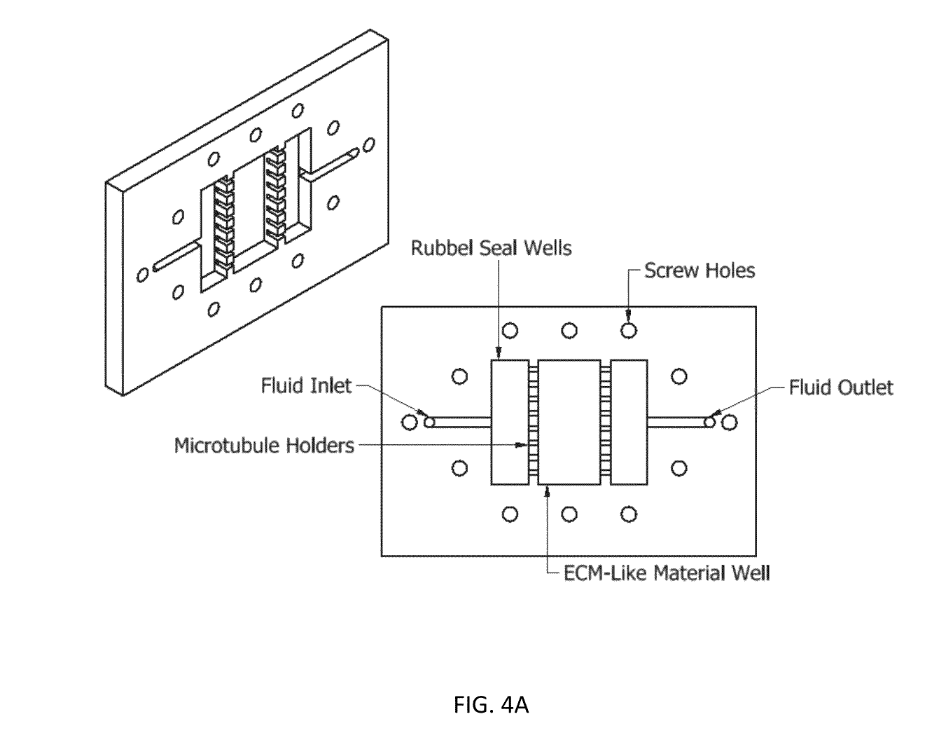

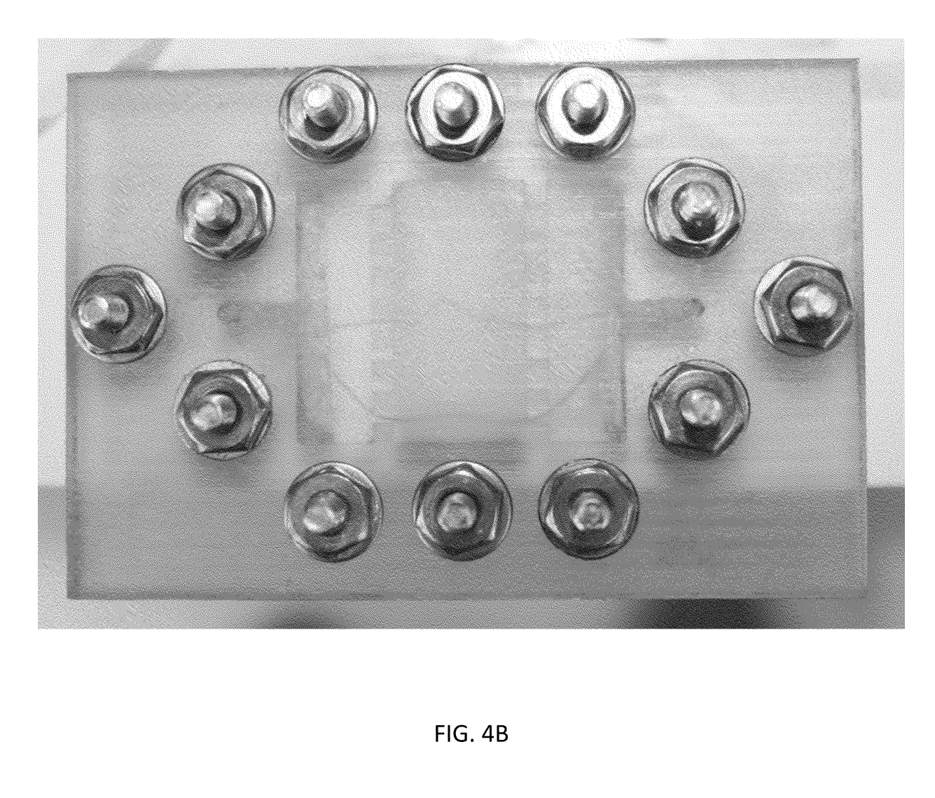

Embodiments of a vascularized organ-on-a-chip microdevice are shown in FIGS. 4A and 4B. The vascularized organ-on-a-chip microdevice has three chambers: an inlet distribution reservoir, an outlet distribution reservoir, and an culture chamber. A series of microtubes with both ends embedded in a manifold connects the inlet and outlet distribution reservoirs across the culture chamber. The culture chamber may include a 3D bio-derived extracellular matrix. The distribution reservoirs include an inlet port and an outlet port for flow of the culture medium. Not shown is a computer-controlled peristaltic pump coupled to the inlet and outlet ports.

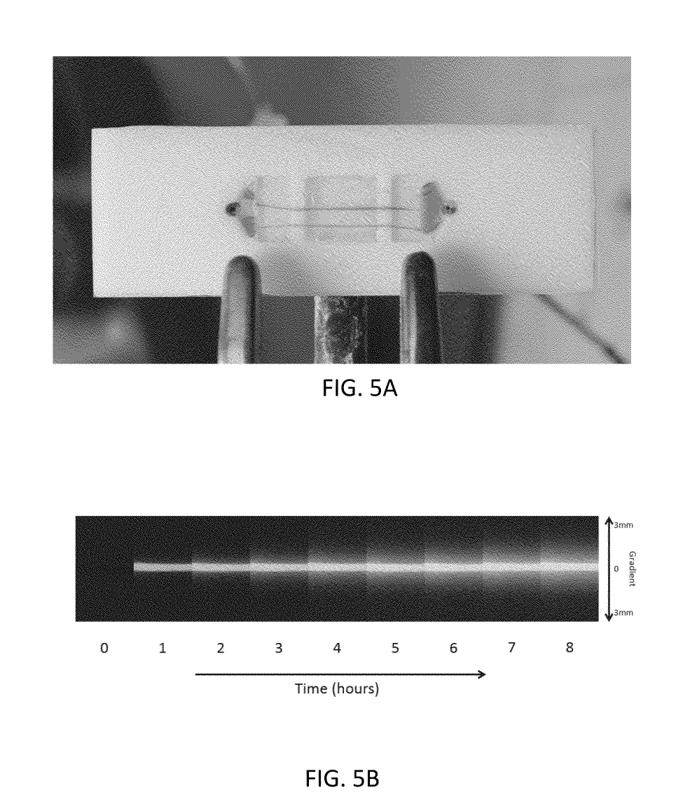

These techniques were validated based on the experimental operation and determination of diffusion across the microtubes in a vascularized organ-on-a-chip microdevice (FIGS. 5A and 5B). The microtube manifold was able to deliver culture medium to the culture chamber by diffusion of fluid across the microtube wall.

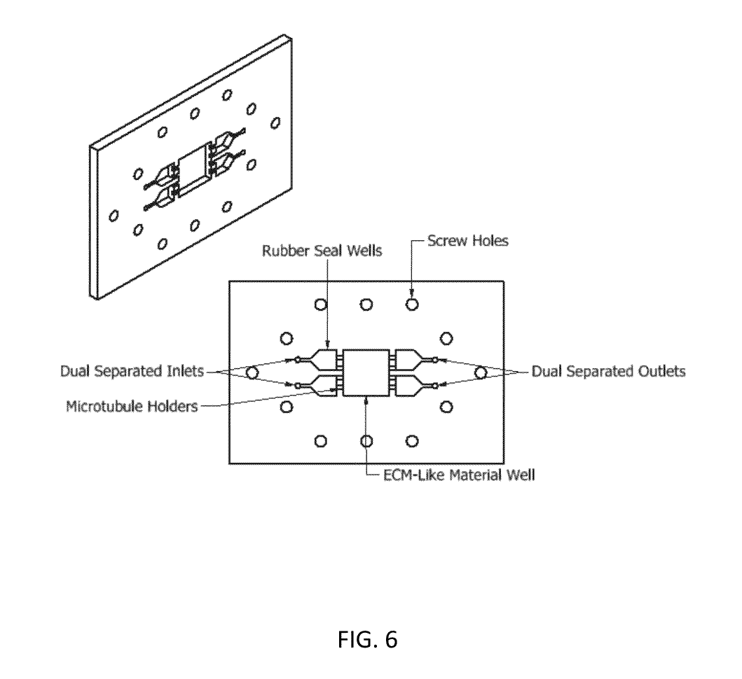

In another embodiment, to measure physiological events in a number of chambers of the tissue-on-chip device in parallel, the device contains more than one inlet and outlet distributions reservoirs, in which the unified culture chamber can be supplied with different materials from isolated distribution chambers (FIG. 6). A similar configuration can be used to characterize the impact of gradients of factors introduced through the microtubes.

A system comprising model microtube networks can include pumps, sensors, controllers, and the like. Sensors can serve to monitor tissue function (for example, pH, electrical conductivity or activity) and can produce imaging data, e.g. using a camera or microscope imaging system, or through an ultrasound or acoustic monitor. Sensors can be connected to perfused flow from the microtubes and/or chamber to monitor analytes such as hormones, proteins (generally or a specific protein of interest), oxygen or other dissolved gases such as nitric oxide, minerals, metabolites, and the like, for example glucose or lactate. Variably pumped flows can simulate blood pumped by a heart or air intake by the lungs. The chamber could also incorporate strain devices to induce periodic mechanical motion as would be experienced by muscles or the heart, for example using mechanical actuators or pneumatic or hydraulic force. The chamber and/or microtubes can also be subjected to electric fields, magnetic fields, and/or irradiation. Valves and pumps can be used to controllable change fluids which are flowed in aspects of the network, including liquids and/or gasses. Typical flows might include liquid nutrient medium optionally supplemented with and without a substance to be studied. For example, the effects of growth factors on cellular proliferation can be studied.

Angiogenesis and anastomosis have been observed in model microtube networks made as described herein. These processes emanated from synthetic blood vessels after their integration into a given tissue and are expected to allow the sustainment of larger scale tissue volumes. It is expected that they can facilitate organ/tissue engineering. Angiogenesis and anastomosis might be controlled as desired by selective perfusion of appropriate factors through the microtubes and/or the chamber. Moreover, model microtube networks might also be used to study these processes.

The proposed vascularized organ-on-a-chip model improves upon many 3D culture methods currently being used in research. It implements free standing microtubes (or alternately microtubes that require support) with customizable and predictable pore size, lumen diameter, and wall thickness. The technique do not require microtube fibers to be made by any particular method and can accommodate those that are relatively soft. By varying the chemistries of the monomers, as well as the hydrodynamic flow used during synthesis of the tubules, molecular size cut offs can be implemented, and predetermined diffusion rates can be implemented. Furthermore, embodiments with multiple inlet and outlet distribution reservoirs allows both the influx and efflux of nutrients and waste, respectively.

Suitable materials include material matrices that compose vascularized tissue. The material that forms the microtube or extracellular matrix in the culture chamber can be composed of any polymerizable material, including but not limited to, synthetic monomers like poly(ethylene glycol) variants, or mammalian derived, functionalized monomers such as polysaccharides (i.e. alginates, hyaluronic acid) or proteins (gelatins, collagen), and combinations thereof. While the microtubes presented in this form are hollow, they can be formed by any number of layers to customize vessels that make up particular tissues. Tubule synthesis can be modified to alter porosity, lumen diameter, or wall thickness. The inclusion of cells in the cladding and/or core synthesis is also possible, thereby providing for the fabrication of cell laden microtubules representative of true in vivo systems for inclusion in the manifold system. The ECM-like matrix can also be customized to provide viscosity and porosity matching the native environment of the modeled tissue. The number of microtubes, inlet and outlet distribution reservoirs are all variable.

Possible types of cells that can be put in the 3D matrix include but are not limited to mesenchymal stem cells, adipose derived stem cells, smooth muscle cells, fibroblasts, hepatocytes, neurons, astrocytes, endothelial cells, epithelial cells, induced pluripotent stem cells, other cell types normally grown in 3D, and combinations thereof. The cells may be prokaryotic or eukaryotic and include, for example, mammalian cells, such as human cells, or plant cells. Bacteria or viruses could be included, for example, to model infection of human tissue.

Possible applied stresses to the device for testing: pneumatic or physical stretching added across chip, magnetic fields, electric fields, introduction of liquid chemicals into the microvessels, introduction of gases in the microvessels.

Concluding Remarks

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

What is claimed is:

1. A model microtube network comprising:
at least one microtube comprising a permeable wall surrounding a central hollow lumen open at opposing ends of the microtube, wherein the microtube has an outer diameter of between 5 and 8000 microns and the permeable wall has a thickness of between 0.1 and 250 microns with a pore size of from 0.1 microns to 500 microns; and
a microfluidic chip having a chamber space defined by chamber walls,
wherein the at least one microtube passes through the chamber space and through the chamber walls with the lumen open at each end outside the chamber space.

2. The model microtube network of claim 1, wherein the microfluidic chip further comprises an input reservoir and an output reservoir each separate from the chamber space and connected to one another by the at least one microtube, and the lumens of the at least one microtube is open at each end to the input reservoir and the output reservoir, respectively.

3. The model microtube network of claim 1, further comprising living animal cells disposed within the permeable wall of the microtube.

4. The model microtube network of claim 1, wherein the permeable wall of the microtube comprises at least two concentric layers of polymer, each layer having a thickness of between 0.1 and 250 microns.

5. The model microtube network of claim 1, wherein said chamber space holds extracellular matrix material.

6. The model microtube network of claim 5, further comprising living animal cells disposed within said extracellular matrix material.

7. The model microtube network of claim 1, wherein said chamber space comprises at least one port for perfusion thereof.

8. A model microtube network comprising:
at least one microtube comprising a permeable wall with living animal cells disposed within, the permeable wall surrounding a central hollow lumen open at opposing ends of the microtube, wherein the microtube has an outer diameter of between 5 and 8000 microns and the permeable wall has a thickness of between 0.1 and 250 microns with a pore size of from 0.1 microns to 500 microns; and
a microfluidic chip having an input reservoir, an output reservoir, and a chamber space therebetween which holds extracellular matrix material, the chamber space being defined by impermeable chamber walls separating the chamber space from the reservoirs,
wherein the at least one microtube passes through the chamber space and through the chamber walls such that a seal exists between the least one microtube and the chamber walls, with the lumen open at each end to the input reservoir and output reservoir, respectively.

9. A method of making a model microtube network, the method comprising:
providing at least one microtube comprising a permeable wall surrounding a central hollow lumen open at opposing ends of the microtube, wherein the microtube has an outer diameter of between 5 and 8000 microns and the permeable wall has a thickness of between 0.1 and 250 microns with a pore size of from 0.1 microns to 500 microns; and
securing the at least one microtube in a microfluidic chip having a chamber space defined by chamber walls, such that the at least one microtube passes through the chamber space and through the chamber walls with the lumen open at each end outside the chamber space.

10. The method of claim 9, wherein said at least one microtube further comprises living animal cells disposed within said permeable wall.

11. The method of claim 9, wherein the microfluidic chip further comprises an input reservoir and an output reservoir each separate from the chamber space and connected to one another by the at least one microtube, and the lumens of the at least one microtube is open at each end to the input reservoir and output reservoir.

12. The method of claim 9, wherein said at least one microtube is prepared by hydrodynamic focusing.

13. The method of claim 9, further comprising sealing the at least one microtube at the passages through the chamber walls such that the chamber walls are impermeable.

14. The method of claim 9, further comprising providing living cells and extracellular matrix in the chamber.

15. A model microtube network system comprising:
model microtube network comprising: at least one microtube comprising a permeable wall surrounding a central hollow lumen open at opposing ends of the microtube, wherein the microtube has an outer diameter of between 5 and 8000 microns and the permeable wall has a thickness of between 0.1 and 250 microns with a pore size of from 0.1 microns to 500 microns; and a microfluidic chip having a chamber space defined by chamber walls, wherein the at least one microtube passes through the chamber space and through the chamber walls with the lumen open at each end outside the chamber space; and
a pump operably connected to cause a fluid flow through the at least one microtube.

16. The system of claim 15, further comprising a sensor.

17. The system of claim 15, further comprising living cells and extracellular matrix in the chamber and/or living cells in the microtube walls.

18. The system of claim 15, wherein the microfluidic chip further comprises an input reservoir and an output reservoir each separate from the chamber space and connected to one another by the at least one microtube, and the lumens of the at least one microtube is open at each end to the input reservoir and output reservoir
